SEQUENCE LISTING (82 sequences)

SEQ ID NO: 1 - RNA, 22 nt, Homo sapiens
ugagguaguagguugugugguu

SEQ ID NO: 2 - RNA, 22 nt, Homo sapiens
ugagguaguaguuuguacaguu

SEQ ID NO: 3 - RNA, 22 nt, Homo sapiens
ugagguaguaguuugugcuguu

SEQ ID NO: 4 - RNA, 22 nt, Homo sapiens
uuuccggcucgcgugggugugu

SEQ ID NO: 5 - RNA, 20 nt, Homo sapiens
ucacuguucagacaggcgga

SEQ ID NO: 6 - RNA, 19 nt, Homo sapiens
gugaggacucgggaggugg

SEQ ID NO: 7 - RNA, 22 nt, Homo sapiens
ugagccccugugccgcccccag

SEQ ID NO: 8 - RNA, 22 nt, Homo sapiens
ucaccagcccuguguucccuag

SEQ ID NO: 9 - RNA, 26 nt, Homo sapiens
gugagggcaugcaggccuggaugggg

SEQ ID NO: 10 - RNA, 20 nt, Homo sapiens
ugagcccuguccucccgcag

SEQ ID NO: 11 - RNA, 22 nt, Homo sapiens
aacuggaucaauuauaggagug

SEQ ID NO: 12 - RNA, 22 nt, Homo sapiens
acccgucccguucguccccgga

SEQ ID NO: 13 - RNA, 22 nt, Homo sapiens
acgcccuucccccccuucuuca

SEQ ID NO: 14 - RNA, 17 nt, Homo sapiens
gugggggagaggcuguc

SEQ ID NO: 15 - RNA, 22 nt, Homo sapiens
acucggcguggcgucggucgug

SEQ ID NO: 16 - RNA, 22 nt, Homo sapiens
uucacauugugcuacugucugc

SEQ ID NO: 17 - RNA, 21 nt, Homo sapiens
uaccacaggguagaaccacgg

SEQ ID NO: 18 - RNA, 22 nt, Homo sapiens
caucuuccaguacaguguugga

SEQ ID NO: 19 - RNA, 22 nt, Homo sapiens
ggugcagugcugcaucucuggu

SEQ ID NO: 20 - RNA, 22 nt, Homo sapiens
ugagaacugaauuccauggguu

SEQ ID NO: 21 - RNA, 22 nt, Homo sapiens
ucgugucuuguguugcagccgg

SEQ ID NO: 22 - RNA, 23 nt, Homo sapiens
caacggaaucccaaaagcagcug

SEQ ID NO: 23 - RNA, 22 nt, Homo sapiens
gcugcgcuuggauuucgucccc

SEQ ID NO: 24 - RNA, 22 nt, Homo sapiens
aacuggccuacaaagucccagu

SEQ ID NO: 25 - RNA, 23 nt, Homo sapiens
cccaguguuuagacuaucuguuc

SEQ ID NO: 26 - RNA, 23 nt, Homo sapiens
ugugcaaaucuaugcaaaacuga

SEQ ID NO: 27 - RNA, 22 nt, Homo sapiens
uaauacugccugguaaugauga

SEQ ID NO: 28 - RNA, 22 nt, Homo sapiens
caucuuacugggcagcauugga

SEQ ID NO: 29 - RNA, 22 nt, Homo sapiens
cugugcgugugacagcggcuga

SEQ ID NO: 30 - RNA, 23 nt, Homo sapiens
agcuacauugucugcuggguuuc

SEQ ID NO: 31 - RNA, 22 nt, Homo sapiens
accuggcauacaauguagauuu

SEQ ID NO: 32 - RNA, 21 nt, Homo sapiens
caagucacuagugguuccguu

SEQ ID NO: 33 - RNA, 22 nt, Homo sapiens
uggguuccuggcaugcugauuu

SEQ ID NO: 34 - RNA, 22 nt, Homo sapiens
cauugcacuugucucggucuga

SEQ ID NO: 35 - RNA, 22 nt, Homo sapiens
uagcaccaucugaaaucgguua

SEQ ID NO: 36 - RNA, 23 nt, Homo sapiens
cagugcaauaguauugucaaagc

SEQ ID NO: 37 - RNA, 17 nt, Homo sapiens
uaauugcuuccauguuu

SEQ ID NO: 38 - RNA, 22 nt, Homo sapiens
cuuucagucggauguuuacagc

SEQ ID NO: 39 - RNA, 22 nt, Homo sapiens
aaaagcuggguugagagggcga

SEQ ID NO: 40 - RNA, 22 nt, Homo sapiens
aaaagcuggguugagagggcaa

SEQ ID NO: 41 - RNA, 20 nt, Homo sapiens
aaaagcuggguugagagggu

SEQ ID NO: 42 - RNA, 20 nt, Homo sapiens
acugccccaggugcugcugg

SEQ ID NO: 43 - RNA, 23 nt, Homo sapiens
cgcauccccuagggcauuggugu

SEQ ID NO: 44 - RNA, 23 nt, Homo sapiens
ugagcgccucgacgacagagccg

SEQ ID NO: 45 - RNA, 23 nt, Homo sapiens
ucccuguccuccaggagcucacg

SEQ ID NO: 46 - RNA, 23 nt, Homo sapiens
ucucacacagaaaucgcacccgu

SEQ ID NO: 47 - RNA, 22 nt, Homo sapiens
gcugacuccuaguccagggcuc

SEQ ID NO: 48 - RNA, 22 nt, Homo sapiens
aaucacuaaccacacggccagg

SEQ ID NO: 49 - RNA, 22 nt, Homo sapiens
uuaucagaaucuccagggguac

SEQ ID NO: 50 - RNA, 22 nt, Homo sapiens
aauugcacgguauccaucugua

SEQ ID NO: 51 - RNA, 22 nt, Homo sapiens
uuauaauacaaccugauaagug

SEQ ID NO: 52 - RNA, 22 nt, Homo sapiens
auauaauacaaccugcuaagug

SEQ ID NO: 53 - RNA, 21 nt, Homo sapiens
aacauagaggaaauuccacgu

SEQ ID NO: 54 - RNA, 21 nt, Homo sapiens
acuggacuuggagucagaagg

SEQ ID NO: 55 - RNA, 22 nt, Homo sapiens
cuccugacuccagguccugugu

SEQ ID NO: 56 - RNA, 23 nt, Homo sapiens
acuucaccugguccacuagccgu

SEQ ID NO: 57 - RNA, 23 nt, Homo sapiens
agcucggucugaggccccucagu

SEQ ID NO: 58 - RNA, 23 nt, Homo sapiens
ugaggggcagagagcgagacuuu

SEQ ID NO: 59 - RNA, 22 nt, Homo sapiens
acccuaucaauauugucucugc

SEQ ID NO: 60 - RNA, 22 nt, Homo sapiens
uaugugccuuuggacuacaucg

SEQ ID NO: 61 - RNA, 22 nt, Homo sapiens
aagacgggaggaaagaagggag

SEQ ID NO: 62 - RNA, 22 nt, Homo sapiens
aguggggaacccuuccaugagg

SEQ ID NO: 63 - RNA, 22 nt, Homo sapiens
augcaccugggcaaggauucug

SEQ ID NO: 64 - RNA, 22 nt, Homo sapiens
aaugcaccugggcaaggauuca

SEQ ID NO: 65 - RNA, 22 nt, Homo sapiens
aucgugcaucccuuuagagugu

SEQ ID NO: 66 - RNA, 22 nt, Homo sapiens
cucuagagggaagcgcuuucug

SEQ ID NO: 67 - RNA, 20 nt, Homo sapiens
cuacaaagggaagcccuuuc

SEQ ID NO: 68 - RNA, 23 nt, Homo sapiens
gaacgcgcuucccuauagagggu

SEQ ID NO: 69 - RNA, 20 nt, Homo sapiens
auguauaaauguauacacac

SEQ ID NO: 70 - RNA, 22 nt, Homo sapiens
cacgcucaugcacacacccaca

SEQ ID NO: 71 - RNA, 23 nt, Homo sapiens
ugagugugugugugugagugugu

SEQ ID NO: 72 - RNA, 25 nt, Homo sapiens
gcugggcagggcuucugagcuccuu

SEQ ID NO: 73 - RNA, 23 nt, Homo sapiens
cuaauaguaucuaccacaauaaa

SEQ ID NO: 74 - RNA, 21 nt, Homo sapiens
aauggcgccacuaggguugug

SEQ ID NO: 75 - RNA, 22 nt, Homo sapiens
ugcggggcuagggcuaacagca

SEQ ID NO: 76 - RNA, 22 nt, Homo sapiens
ugagaccucuggguucugagcu

SEQ ID NO: 77 - RNA, 22 nt, Homo sapiens
cugcccuggcccgagggaccga

SEQ ID NO: 78 - RNA, 21 nt, Homo sapiens
uuaauaucggacaaccauugu

SEQ ID NO: 79 - RNA, 22 nt, Homo sapiens
uauugcacucgucccggccucc

SEQ ID NO: 80 - RNA, 22 nt, Homo sapiens
acaguagagggaggaaucgcag

SEQ ID NO: 81 - RNA, 22 nt, Homo sapiens
cacccguagaaccgaccuugcg

SEQ ID NO: 82 - RNA, 22 nt, Homo sapiens
caagcucgugucuguggguccg

(19) United States
(12) Patent Application Publication
(10) Pub. No.: US 2015/0080243 A1
(43) Pub. Date: Mar. 19, 2015

(54) METHODS AND COMPOSITIONS FOR DETECTING CANCER BASED ON MIRNA EXPRESSION PROFILES

(71) Applicants: Duncan H. Whitney, Boston, MA (US); Jun Luo, Belmont, MA (US)

(72) Inventors: Duncan H. Whitney, Boston, MA (US); Jun Luo, Belmont, MA (US)

(73) Assignee: ALLEGRO DIAGNOSTICS CORP., Maynard, MA (US)

(21) Appl. No.: 14/342,491

(22) PCT Filed: Aug. 31, 2012
(86) PCT No.: PCT/US2012/053531

§ 371 Date: Dec. 1, 2014

Related U.S. Application Data
(60) Provisional application No. 61/530,235, filed on Sep. 1, 2011.

(51) Int. Cl. C12Q 1/68; G06F 19/18

(52) U.S. Cl. CPC C12Q 1/6886; G06F 19/18; C12Q 2600/178; C12Q 2600/158; C12Q 2600/118
USPC 506/9; 702/19

(57) ABSTRACT

The disclosure in some aspects provides methods of determining the likelihood that a subject has lung cancer based on the expression of informative miRNAs. In other aspects, the disclosure provides methods for determining a treatment course for a subject based on the expression of informative-miRNAs. The disclosure also provides computer implemented methods for processing genomic information relating to miRNA expression. Related compositions and kits are provided in other aspects of the disclosure.

### RELATED APPLICATION

This application claims priority under 35 U.S.C. §119 to U.S. provisional patent application, U.S. Ser. No. 61/530,235, filed Sep. 1, 2011, entitled “Methods and Compositions for Detecting Cancer Based on miRNA Expression Profiles,” the entire contents of which are incorporated herein by reference.

### FIELD

The present disclosure, at least in some aspects, generally relates to methods and compositions for assessing cancer risk using genomic information.

### BACKGROUND

A challenge in diagnosing lung cancer, particularly at an early stage where it can be most effectively treated, is gaining access to cells to diagnose disease. Early stage lung cancer is typically associated with small lesions, which may also appear in the peripheral regions of the lung airway, which are particularly difficult to reach by standard techniques such as bronchoscopy.

### SUMMARY

Provided herein are methods for determining the likelihood that a subject has lung cancer. In some embodiments, methods involve making a risk assessment based on expression levels of informative-miRNAs in a biological sample obtained from the subject during a routine cell or tissue sampling procedure. Methods described herein can be used to assess the likelihood that an individual has lung cancer by evaluating histologically normal cells or tissues obtained during a routine cell or tissue sampling procedure (e.g., standard ancillary bronchoscopic procedures such as brushing, biopsy, lavage, and needle-aspiration). However, it should be appreciated that any suitable tissue or cell sample can be used. In some embodiments, the cells or tissues that are assessed by the methods provided herein appear histologically normal. Some methods described herein, alone or in combination with other methods, provide useful information for health care providers to assist them in making diagnostic and therapeutic decisions for a patient. In some embodiments, methods disclosed herein are employed in instances where other methods have failed to provide useful information regarding the lung cancer status of a patient. Some of the methods disclosed herein provide an alternative or complementary method for evaluating or diagnosing cell or tissue samples obtained during routine bronchoscopy procedures, and increase the likelihood that the procedures will result in useful information for managing a patient's care. The methods disclosed herein are highly sensitive, and produce information regarding the likelihood that a subject has lung cancer from cell or tissue samples (e.g., histologically normal tissue) that may be obtained from positions remote from malignant lung tissue. Methods are provided, in some embodiments, for obtaining biological samples from patients. Expression levels of informative-miRNAs in these biological samples provide a basis for assessing the likelihood that the patient has lung cancer. Methods are provided for processing biological samples. In general, the processing methods ensure RNA quality and integrity to enable downstream analysis of informative-miRNAs and ensure quality in the results obtained. Accordingly, various quality control steps (e.g., RNA size analyses) may be employed in these methods. Methods are provided for packaging and storing biological samples. Methods are provided for shipping or transporting biological samples, e.g., to an assay laboratory where the biological sample may be processed and/or where a gene expression analysis may be performed. Methods are provided for performing gene expression analyses on biological samples to determine the expression levels of informative-miRNAs in the samples. Methods are provided for analyzing and interpreting the results of gene expression analyses of informative-miRNAs. Methods are provided for generating reports that summarize the results of gene expression analyses, and for transmitting or sending assay results and/or assay interpretations to a health care provider (e.g., a physician). Furthermore, methods are provided for making treatment decisions based on the gene expression assay results, including making recommendations for further treatment or invasive diagnostic procedures.

Some aspects of this disclosure are based, at least in part, on the determination that the expression level of one or more miRNA molecules in apparently histologically normal cells obtained from a first airway locus can be used to evaluate the likelihood of cancer at a second locus in the airway (for example, at a locus in the airway that is remote from the locus at which the histologically normal cells were sampled).

Some aspects of this disclosure relate to determining the likelihood that a subject has lung cancer, by subjecting a biological sample obtained from a subject to a gene expression analysis, wherein the gene expression analysis comprises determining expression levels in the biological sample of at least two miRNAs selected from Table 6, and using the expression levels to assist in determining the likelihood that the subject has or will develop lung cancer.

In some embodiments, the step of determining comprises transforming the expression levels into a lung cancer risk-score that is indicative of the likelihood that the subject has lung cancer. In some embodiments, the lung cancer risk-score is the combination of weighted expression levels. In some embodiments, the lung cancer risk-score is the sum of weighted expression levels. In some embodiments, the expression levels are weighted by their relative contribution to predicting increased likelihood of having lung cancer.

Some aspects of this disclosure relate to determining a treatment course for a subject, by subjecting a biological sample obtained from the subject to a gene expression analysis, wherein the gene expression analysis comprises determining the expression levels in the biological sample of at least two miRNAs selected from Table 6, and determining a treatment course for the subject based on the expression levels.

In some embodiments, the treatment course is determined based on a lung cancer risk-score derived from the expression levels. In some embodiments, the subject is identified as a candidate for a lung cancer therapy based on a lung cancer risk-score that indicates the subject has a relatively high likelihood of having lung cancer. In some embodiments, the subject is identified as a candidate for an invasive lung procedure based on a lung cancer risk-score that indicates the subject has a relatively high likelihood of having lung cancer. In some embodiments, the invasive lung procedure comprises a transthoracic needle aspiration, mediastinoscopy, lobectomy, or thoracotomy. In some embodiments, the subject is identified as not being a candidate for a lung cancer therapy or an invasive lung procedure based on a lung cancer risk-score that indicates the subject has a relatively low likelihood of having lung cancer. In some embodiments, a report summarizing the results of the gene expression analysis is created. In some embodiments, the report indicates the lung cancer risk-score.

Some aspects of this disclosure relate to determining the likelihood that a subject has lung cancer by subjecting a biological sample obtained from a subject to a gene expression analysis, wherein the gene expression analysis comprises determining the expression levels in the biological sample of at least one miRNA selected from Table 6 other than miR-221, and determining the likelihood that the subject has lung cancer based at least in part on the expression levels.

Some aspects of this disclosure relate to determining the likelihood that a subject has lung cancer, by subjecting a biological sample obtained from the respiratory epithelium of a subject to a gene expression analysis, wherein the gene expression analysis comprises determining the expression level in the biological sample of at least one miRNA selected from Table 6, and determining the likelihood that the subject has lung cancer based at least in part on the expression level, wherein the biological sample comprises histologically normal tissue.

Some aspects of this disclosure relate to a computer-implemented method for processing genomic information, by obtaining data representing expression levels in a biological sample of at least two miRNAs selected from Table 6, wherein the biological sample was obtained of a subject, and using the expression levels to assist in determining the likelihood that the subject has lung cancer. A computer-implemented method can include inputting data via a user interface, computing (e.g., calculating, comparing, or otherwise analyzing) using a processor, and/or outputting results via a display or other user interface.

In some embodiments, the step of determining comprises calculating a risk-score indicative of the likelihood that the subject has lung cancer. In some embodiments, computing the risk-score involves determining the combination of weighted expression levels, wherein the expression levels are weighted by their relative contribution to predicting increased likelihood of having lung cancer. In some embodiments, a computer-implemented method comprises generating a report that indicates the risk-score. In some embodiments, the report is transmitted to a health care provider of the subject.

It should be appreciated that in any embodiment or aspect described herein, a biological sample can be obtained from the respiratory epithelium of the subject. The respiratory epithelium can be of the mouth, nose, pharynx, trachea, bronchi, bronchioles, or alveoli. However, other sources of respiratory epithelium also can be used. The biological sample can comprise histologically normal tissue. The biological sample can be obtained using bronchial brushings, broncho-alveolar lavage, or a bronchial biopsy. The subject can exhibit one or more symptoms of lung cancer and/or have a lesion that is observable by computer-aided tomography or chest X-ray. In some cases, prior to subjecting the biological sample of a subject to a gene expression analysis, the subject has not be diagnosed with primary lung cancer.

It also should be appreciated that in any of the embodiments or aspects described herein at least two miRNAs can be selected from the group consisting of: hsa-miR-210; hsa-miR-378; hsa-miR-221*; hsa-miR-320b; hsa-miR-1226*; hsa-miR-744; hsa-miR-320a; hsa-miR-1243; hsa-miR-345; and hsa-miR-200b. For example, the at least two miRNAs can be selected from the group consisting of: hsa-miR-210; hsa-miR-378; hsa-miR-221*; hsa-miR-320b; and hsa-miR-1226*, or the group consisting of: hsa-miR-210; hsa-miR-378; and hsa-miR-221*. The gene expression analysis can comprise determining the expression levels in the RNA sample of at least five miRNAs selected from Table 6, or at least ten miRNAs selected from Table 6.

In any of the embodiments or aspects described herein, the expression levels can be determined using a quantitative reverse transcription polymerase chain reaction, a bead-based nucleic acid detection assay or a oligonucleotide array assay, or any other suitable assay.

In any of the embodiments or aspects described herein, the lung cancer can be a adenocarcinoma, squamous cell carcinoma, small cell cancer or non-small cell cancer.

Some aspects of this disclosure relate to a composition consisting essentially of at least two nucleic acid probes, wherein each of the at least two nucleic acids probes specifically hybridizes with an miRNA selected from Table 6.

Some aspects of this disclosure relate to a composition comprising up to 5, up to 10, up to 25, up to 50, up to 100, or up to 200 nucleic acid probes, wherein each of at least two of the nucleic acid probes specifically hybridizes with an miRNA selected from Table 6. In some embodiments, one or more (e.g., 2, 3, 4, 5, or more) miRNAs described herein are excluded from an assay.

In some embodiments, the miRNA is selected from the group consisting of: hsa-miR-210; hsa-miR-378; hsa-miR-221*; hsa-miR-320b; hsa-miR-1226*; hsa-miR-744; hsa-miR-320a; hsa-miR-1243; hsa-miR-345; and hsa-miR-200b. In some embodiments, the miRNA is selected from the group consisting of: hsa-miR-210; hsa-miR-378; hsa-miR-221*; hsa-miR-320b; and hsa-miR-1226*. In some embodiments, the miRNA is selected from the group consisting of: hsa-miR-210; hsa-miR-378; and hsa-miR-221*. In some embodiments, each of at least five of the nucleic acid probes specifically hybridizes with an miRNA selected from Table 6 or with a nucleic acid having a sequence complementary to the miRNA. In some embodiments, each of at least ten of the nucleic acid probes specifically hybridizes with an miRNA selected from Table 6 or with a nucleic acid having a sequence complementary to the miRNA.

In some embodiments, the nucleic acid probes are conjugated directly or indirectly to a bead. In some embodiments, the bead is a magnetic bead. In some embodiments, the nucleic acid probes are immobilized to a solid support. In some embodiments, the solid support is a glass, plastic or silicon chip.

Some aspects of this disclosure relate to a kit comprising at least one container or package housing any nucleic acid probe composition described herein.

Some aspects of this disclosure relate to a method of processing an RNA sample, by obtaining an RNA sample, determining the expression level of a first miRNA in the RNA sample, and determining the expression level of a second miRNA in the RNA sample, wherein the expression level of the first miRNA and the second miRNA are determined in biochemically separate assays, and wherein the first miRNA and second miRNA are selected from Table 6.

In some embodiments, the expression level of at least one other miRNA is determined in the RNA sample, wherein the expression level of the first miRNA, the second miRNA, and the at least one other miRNA are determined in biochemically separate assays, and wherein the at least one other miRNA is selected from Table 6.

In some embodiments, expression levels are determined using a quantitative reverse transcription polymerase chain reaction.

These and other aspects are described in more detail herein and are illustrated by the non-limiting figures and examples.

### BRIEF DESCRIPTION OF DRAWINGS

FIG. 1 depicts the results of a principal component analysis on miRNA expression levels obtained for all 30 cancers and 30 no-cancers, showing that the majority of samples cluster together);

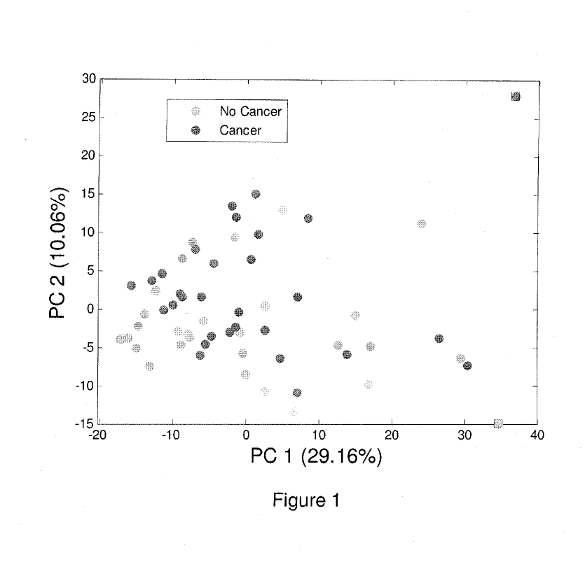

FIG. 2 depicts a heatmap that is separated to illustrate miRNAs up-regulated (positive values on the scale at the right) in versus those down-regulated (negative values on the scale) in both cancer and no-cancer subjects; and

FIG. 3 depicts the results of a Monte-Carlo cross-validation approach that was used to assign samples to separate training and test sets, whereby the accuracy of the prediction model was recorded (in this case using sensitivity, specificity, accuracy, and area under the curve (AUC) of a receiver operator characteristic (ROC) curve) as a function of the number of miRNAs selected in the biomarker. Prediction accuracy was determined using an SVM classifier.

### DETAILED DESCRIPTION

Provided herein are methods for determining the likelihood that a subject has lung cancer, such as adenocarcinoma, squamous cell carcinoma, small cell cancer or non-small cell cancer. Some of the methods provided herein, alone or in combination with other methods, provide useful information for health care providers to assist them in making diagnostic and therapeutic decisions for a patient. In some embodiments, methods disclosed herein are employed in instances where other methods have failed to provide useful information regarding the lung cancer status of a patient. For example, approximately 50% of bronchoscopy procedures result in indeterminate or non-diagnostic information. There are multiple sources of indeterminate results, and the outcome of bronchoscopy (e.g., diagnostic v. non-diagnostic) may depend on the training and procedures available at different medical centers. In some embodiments, methods provided herein are employed to determine the likelihood that a subject has lung cancer after the subject has been subjected to a bronchoscopy. In some embodiments, methods provided herein are employed to provide an indication of whether or not a subject has lung cancer after the subject has been subjected to a bronchoscopy and the result of the bronchoscopy was non-diagnostic.

Methods disclosed herein provide alternative or complementary approaches for evaluating cell or tissue samples obtained by bronchoscopy procedures (or other procedures for evaluating respiratory tissue), and increase the likelihood that the procedures will result in useful information for managing the patient's care. The methods disclosed herein are highly sensitive, and produce information regarding the likelihood that a subject has lung cancer from cell or tissue samples (e.g., bronchial brushings of airway epithelial cells), for example, from cell or tissue samples obtained from regions in the airway that are remote from malignant lung tissue. In some embodiments, methods are disclosed herein that involve subjecting a biological sample obtained from a subject, for example, a cell or tissue sample obtained from bronchial brushings, to a gene expression analysis to evaluate miRNA expression levels. In some embodiments, the likelihood that the subject has lung cancer is determined based on the results of a histological examination of the biological sample or by considering other diagnostic indicia such as protein levels, mRNA levels, imaging results, chest X-ray exam results etc.

The term “subject,” as used herein, generally refers to a mammal. Typically the subject is a human. However, the term embraces other species, e.g., pigs, mice, rats, dogs, cats, or other primates. In certain embodiments, the subject is an experimental subject such as a mouse or rat. The subject may be a male or female. The subject may be an infant, a toddler, a child, a young adult, an adult or a geriatric. The subject may be a smoker, a former smoker or a non-smoker. The subject may have a personal or family history of cancer. The subject may have a cancer-free personal or family history. The subject may exhibit one or more symptoms of lung cancer or other lung disorder (e.g., emphysema, COPD). For example, the subject may have a new or persistent cough, worsening of an existing chronic cough, blood in the sputum, persistent bronchitis or repeated respiratory infections, chest pain, unexplained weight loss and/or fatigue, or breathing difficulties such as shortness of breath or wheezing. The subject may have a lesion, which may be observable by computer-aided tomography or chest X-ray. The subject may be an individual who has undergone a bronchoscopy or who has been identified as a candidate for bronchoscopy (e.g., because of the presence of a detectable lesion or suspicious imaging result). A subject under the care of a physician or other health care provider may be referred to as a “patient.”

### Informative-miRNAs

MiRNAs (also referred to as MicroRNAs) are small non-coding RNAs that regulate mRNA expression post-transcriptionally. The expression levels of the miRNAs in Table 6 have been identified herein as providing useful information regarding the lung cancer status of a subject. These miRNAs are referred to herein as “informative-miRNAs.”

The methods disclosed herein involve determining expression levels in the biological sample of at least one informative-miRNA selected from Table 6. For example, in some embodiments, the expression analysis involves determining the expression levels in the biological sample of at least 2, at least 3, at least 4, at least 5, at least 6, at least 7, at least 8, at least 9, at least 10, at least 20, at least 30, at least 40, at least 50, at least 60, at least 70, or least 80 miRNAs selected from Table 6. In some embodiments, methods disclosed herein involve determining expression levels in the biological sample of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 informative-miRNAs provided herein, e.g., miRNAs selected from Table 6.

In some embodiments, miRNAs (e.g., at least 2, at least 3, at least 4, at least 5, etc.; or 2, 3, 4, 5, 6, 7, 8, 9, or 10 miRNAs) are selected from the group consisting of: hsa-miR-210; hsa-miR-378; hsa-miR-221*; hsa-miR-320b; hsa-miR-1226*; hsa-miR-744; hsa-miR-320a; hsa-miR-1243; hsa-miR-345; and hsa-miR-200b. In some embodiments, miRNAs (e.g., at least 2, at least 3, at least 4, or 2, 3, 4, or 5 miRNAs) are selected from the group consisting of: hsa-miR-210; hsa-miR-378; hsa-miR-221*; hsa-miR-320b; and hsa-miR-1226*. In some embodiments, at least 2 miRNAs (e.g., 2 or 3 miRNAs) are selected from the group consisting of: hsa-miR-210; hsa-miR-378; and hsa-miR-221*. In some embodiments, at least one miRNA is miR221* and at least one other miRNA (e.g., at least one, at least two, at least three, etc., or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 miRNAs) is selected from Table 6. In some embodiments, only informative-miRNAs from Table 6 other than miR-221 are used in the analysis

In some embodiments, the number of miRNAs that are selected from Table 6 for a gene expression analysis are sufficient to provide a level of confidence in a prediction outcome that is clinically useful. This level of confidence (e.g., strength of a prediction model) may be assessed by a variety of performance parameters including, but not limited to, the accuracy, sensitivity specificity, and area under the curve (AUC) of the receiver operator characteristic (ROC). These parameters may be assessed with varying numbers of features (miRNA expression levels) to determine an optimum number and set of miRNAs. An accuracy, sensitivity or specificity of at least 60%, 70%, 80%, 90%, may be useful when used alone or in combination with other information.

Any appropriate system or method may be used for determining expression levels of informative-miRNAs. Typically, miRNA expression levels are determining through the use of a hybridization-based assay. As used herein, the term, “hybridization-based assay” refers to any assay that involves nucleic acid hybridization. A hybridization-based assay may or may not involve amplification of nucleic acids. Hybridization-based assays are well known in the art and include, but are not limited to, array-based assays (e.g., oligonucleotide arrays, microarrays), oligonucleotide conjugated bead assays (e.g., Multiplex Bead-based Luminex® Assays), molecular inversion probe assays, and quantitative RT-PCR assays. Multiplex systems, such as oligonucleotide arrays or bead-based nucleic acid assay systems are particularly useful for evaluating levels of a plurality of miRNA simultaneously. Other appropriate methods for determining levels of nucleic acids will be apparent to the skilled artisan.

As used herein, a “level” refers to a value indicative of the amount or occurrence of a substance, e.g., an miRNA. A level may be an absolute value, e.g., a quantity of an miRNA in a sample, or a relative value, e.g., a quantity of an miRNA in a sample relative to the quantity of the miRNA in a reference sample (control sample). The level may also be a binary value indicating the presence or absence of a substance. For example, a substance may be identified as being present in a sample when a measurement of the quantity of the substance in the sample, e.g., a fluorescence measurement from a PCR reaction or microarray, exceeds a background value. Similarly, a substance may be identified as being absent from a sample (or undetectable in the sample) when a measurement of the quantity of the molecule in the sample is at or below background value. It should be appreciated that the level of a substance may be determined directly or indirectly.

### Biological Samples

The methods generally involve obtaining a biological sample from a subject. As used herein, the phrase “obtaining a biological sample” refers to any process for directly or indirectly acquiring a biological sample from a subject. For example, a biological sample may be obtained (e.g., at a point-of-care facility, a physician's office, a hospital) by procuring a tissue or fluid sample from a subject. Alternatively, a biological sample may be obtained by receiving the sample (e.g., at a laboratory facility) from one or more persons who procured the sample directly from the subject.

The term “biological sample” refers to a sample derived from a subject, e.g., a patient. A biological sample typically comprises a tissue, cells and/or biomolecules. In some embodiments, a biological sample is obtained on the basis that it is histologically normal, e.g., as determined by endoscopy, e.g., bronchoscopy. In some embodiments, the biological sample is a sample of respiratory epithelium. The respiratory epithelium may be of the mouth, nose, pharynx, trachea, bronchi, bronchioles, or alveoli of the subject. The biological sample may comprise epithelium of the bronchi. In some embodiments, the biological sample is free of detectable cancer cells, e.g., as determined by standard histological or cytological methods. In some embodiments, histologically normal samples are obtained for evaluation. Often biological samples are obtained by scrapings or brushings, e.g., bronchial brushings. However, it should be appreciated that other procedures may be used, including, for example, brushings, scrapings, broncho-alveolar lavage, a bronchial biopsy or a transbronchial needle aspiration.

It is to be understood that a biological sample may be processed in any appropriate manner to facilitate determining expression levels. For example, biochemical, mechanical and/or thermal processing methods may be appropriately used to isolate a biomolecule of interest, e.g., RNA, from a biological sample. Accordingly, a RNA or other molecules may be isolated from a biological sample by processing the sample using methods well known in the art.

### Lung Cancer Assessment

Methods disclosed herein may involve comparing expression levels of informative-miRNAs with one or more appropriate references. An “appropriate reference” is an expression level (or range of expression levels) of a particular informative-miRNA that is indicative of a known lung cancer status. An appropriate reference can be determined experimentally by a practitioner of the methods or can be a pre-existing value or range of values. An appropriate reference represent an expression level (or range of expression levels) indicative of lung cancer. For example, an appropriate reference may be representative of the expression level of an informative-miRNA in a reference (control) biological sample obtained from a subject who is known to have lung cancer. When an appropriate reference is indicative of lung cancer, a lack of a detectable difference (e.g., lack of a statistically significant difference) between an expression level determined from a subject in need of characterization or diagnosis of lung cancer and the appropriate reference may be indicative of lung cancer in the subject. When an appropriate reference is indicative of lung cancer, a difference between an expression level determined from a subject in need of characterization or diagnosis of lung cancer and the appropriate reference may be indicative of the subject being free of lung cancer.

Alternatively, an appropriate reference may be an expression level (or range of expression levels) of an miRNA that is indicative of a subject being free of lung cancer. For example, an appropriate reference may be representative of the expression level of a particular informative-miRNA in a reference (control) biological sample obtained from a subject who is known to be free of lung cancer. When an appropriate reference is indicative of a subject being free of lung cancer, a difference between an expression level determined from a subject in need of diagnosis of lung cancer and the appropriate reference may be indicative of lung cancer in the subject. Alternatively, when an appropriate reference is indicative of the subject being free of lung cancer, a lack of a detectable difference (e.g., lack of a statistically significant difference) between an expression level determined from a subject in need of diagnosis of lung cancer and the appropriate reference level may be indicative of the subject being free of lung cancer.

In some embodiments, the reference standard provides a threshold level of change, such that if the expression level of an miRNA in a sample is within a threshold level of change (increase or decrease depending on the particular marker) then the subject is identified as free of lung cancer, but if the levels are above the threshold then the subject is identified as being at risk of having lung cancer.

In some embodiments, the methods involve comparing the expression level of an miRNA to a reference standard that represents the expression level of the miRNA in a control subject who is identified as not having lung cancer. This reference standard may be, for example, the average expression level of the miRNA in a population of control subjects who are identified as not having lung cancer. In these embodiments, increased expression of an miRNA that has a positive weight in the last column of Table 3 or 4, compared with the reference standard, is indicative of the subject having lung cancer. Furthermore, decreased expression of an miRNA that has a negative weight in the last column of Table 3 or 4, compared with the reference standard, is indicative of the subject having lung cancer.

The magnitude of difference between a expression level and an appropriate reference that is statistically significant may vary. For example, a significant difference that indicates lung cancer may be detected when the expression level of an informative-miRNA in a biological sample is at least 1%, at least 5%, at least 10%, at least 25%, at least 50%, at least 100%, at least 250%, at least 500%, or at least 1000% higher, or lower, than an appropriate reference of that miRNA. Similarly, a significant difference may be detected when the expression level of informative-miRNA in a biological sample is at least 1.1-fold, 1.2-fold, 1.5-fold, 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the appropriate reference of that miRNA. Significant differences may be identified by using an appropriate statistical test. Tests for statistical significance are well known in the art and are exemplified in Applied Statistics for Engineers and Scientists by Petruccelli, Chen and Nandram 1999 Reprint Ed.

It is to be understood that a plurality of expression levels may be compared with plurality of appropriate reference levels, e.g., on a miRNA-by-miRNA basis, in order to assess the lung cancer status of the subject. The comparison may be made as a vector difference. In such cases, Multivariate Tests, e.g., Hotelling's T² test, may be used to evaluate the significance of observed differences. Such multivariate tests are well known in the art and are exemplified in Applied Multivariate Statistical Analysis by Richard Arnold Johnson and Dean W. Wichern Prentice Hall; 4^{th} edition (Jul. 13, 1998).

### Classification Methods

The methods may also involve comparing a set of expression levels (referred to as an expression pattern or profile) of informative-miRNAs in a biological sample obtained from a subject with a plurality of sets of reference levels (referred to as reference patterns), each reference pattern being associated with a known lung cancer status, identifying the reference pattern that most closely resembles the expression pattern, and associating the known lung cancer status of the reference pattern with the expression pattern, thereby classifying (characterizing) the lung cancer status of the subject.

The methods may also involve building or constructing a prediction model, which may also be referred to as a classifier or predictor, that can be used to classify the disease status of a subject. As used herein, a “lung cancer-classifier” is a prediction model that characterizes the lung cancer status of a subject based on expression levels determined in a biological sample obtained from the subject. Typically the model is built using samples for which the classification (lung cancer status) has already been ascertained. Once the model (classifier) is built, it may then be applied to expression levels obtained from a biological sample of a subject whose lung cancer status is unknown in order to predict the lung cancer status of the subject. Thus, the methods may involve applying a lung cancer-classifier to the expression levels, such that the lung cancer-classifier characterizes the lung cancer status of a subject based on the expression levels. The subject may be further treated or evaluated, e.g., by a health care provider, based on the predicted lung cancer status.

The classification methods may involve transforming the expression levels into a lung cancer risk-score that is indicative of the likelihood that the subject has lung cancer. In some embodiments, such as, for example, when a linear discriminant classifier is used, the lung cancer risk-score may be obtained as the combination (e.g., sum, product) of weighted expression levels, in which the expression levels are weighted by their relative contribution to predicting increased likelihood of having lung cancer.

It should be appreciated that a variety of prediction models known in the art may be used as a lung cancer-classifier. For example, a lung cancer-classifier may comprises an algorithm selected from logistic regression, partial least squares, linear discriminant analysis, quadratic discriminant analysis, neural network, naïve Bayes, C4.5 decision tree, k-nearest neighbor, random forest, and support vector machine. Other appropriate methods will be apparent to the skilled artisan.

The lung cancer-classifier may be trained on a data set comprising expression levels of the plurality of informative-miRNAs in biological samples obtained from a plurality of subjects identified as having lung cancer. For example, the lung cancer-classifier may be trained on a data set comprising expression levels of a plurality of informative-miRNAs in biological samples obtained from a plurality of subjects identified as having lung cancer based histological findings. The training set will typically also comprise control subjects identified as not having lung cancer. As will be appreciated by the skilled artisan, the population of subjects of the training data set may have a variety of characteristics by design, e.g., the characteristics of the population may depend on the characteristics of the subjects for whom diagnostic methods that use the classifier may be useful. For example, the population may consist of all males, all females or may consist of both males and females. The population may consist of subjects with history of cancer, subjects without a history of cancer, or a subjects from both categories. The population may include subjects who are smokers, former smokers, and/or non-smokers.

A class prediction strength can also be measured to determine the degree of confidence with which the model classifies a biological sample. This degree of confidence may serve as an estimate of the likelihood that the subject is of a particular class predicted by the model. Accordingly, the prediction strength conveys the degree of confidence of the classification of the sample and evaluates when a sample cannot be classified. There may be instances in which a sample is tested, but does not belong, or cannot be reliably assigned to, a particular class. This may be accomplished, for example, by utilizing a threshold, or range, wherein a sample which scores above or below the determined threshold, or within the particular range, is not a sample that can be classified (e.g., a “no call”).

Once a model is built, the validity of the model can be tested using methods known in the art. One way to test the validity of the model is by cross-validation of the dataset. To perform cross-validation, one, or a subset, of the samples is eliminated and the model is built, as described above, without the eliminated sample, forming a “cross-validation model.” The eliminated sample is then classified according to the model, as described herein. This process is done with all the samples, or subsets, of the initial dataset and an error rate is determined. The accuracy the model is then assessed. This model classifies samples to be tested with high accuracy for classes that are known, or classes have been previously ascertained. Another way to validate the model is to apply the model to an independent data set, such as a new biological sample having an unknown lung cancer status.

As will be appreciated by the skilled artisan, the strength of the model may be assessed by a variety of parameters including, but not limited to, the accuracy, sensitivity and specificity. Methods for computing accuracy, sensitivity and specificity are known in the art and described herein (See, e.g., the Examples). The lung cancer-classifier may have an accuracy of at least 60%, at least 65%, at least 70%, at least 75%, at least 80%, at least 85%, at least 90%, at least 95%, at least 99%, or more. The lung cancer-classifier may have an accuracy in a range of about 60% to 70%, 70% to 80%, 80% to 90%, or 90% to 100%. The lung cancer-classifier may have an sensitivity of at least 60%, at least 65%, at least 70%, at least 75%, at least 80%, at least 85%, at least 90%, at least 95%, at least 99%, or more. The lung cancer-classifier may have an sensitivity in a range of about 60% to 70%, 70% to 80%, 80% to 90%, or 90% to 100%. The lung cancer-classifier may have an specificity of at least 60%, at least 65%, at least 70%, at least 75%, at least 80%, at least 85%, at least 90%, at least 95%, at least 99%, or more. The lung cancer-classifier may have an specificity in a range of about 60% to 70%, 70% to 80%, 80% to 90%, or 90% to 100%.

Clinical Treatment/Management

In certain aspects, methods are provided for determining a treatment course for a subject. The methods typically involve determining the expression levels in a biological sample obtained from the subject of one or more informative-miRNAs, and determining a treatment course for the subject based on the expression levels. Often the treatment course is determined based on a lung cancer risk-score derived from the expression levels. The subject may be identified as a candidate for a lung cancer therapy based on a lung cancer risk-score that indicates the subject has a relatively high likelihood of having lung cancer. The subject may be identified as a candidate for an invasive lung procedure (e.g., transthoracic needle aspiration, mediastinoscopy, lobectomy, or thoracotomy) based on a lung cancer risk-score that indicates the subject has a relatively high likelihood of having lung cancer (e.g., greater than 60%, greater than 70%, greater than 80%, greater than 90%). The subject may be identified as not being a candidate for a lung cancer therapy or an invasive lung procedure based on a lung cancer risk-score that indicates the subject has a relatively low likelihood (e.g., less than 50%, less than 40%, less than 30%, less than 20%) of having lung cancer. In some cases, an intermediate risk-score is obtained and the subject is not indicated as being in the high risk or the low risk categories. In some embodiments, a health care provider may engage in “watchful waiting” and repeat the analysis on biological samples taken at one or more later points in time, or undertake further diagnostics procedures to rule out lung cancer, or make a determination that cancer is present, soon after the risk determination was made. The methods may also involve creating a report that summarizes the results of the gene expression analysis. Typically the report would also include an indication of the lung cancer risk-score.

### Drug Discovery

In some embodiments, the present disclosure provides methods for identifying miRNAs that are associated with therapeutically targetable pathways, or are themselves part of a therapeutically targetable pathway. In these embodiments, candidate therapeutic compounds may impact the expression of miRNAs and reverse the observed disease-related differential expression of one or more miRNAs disclosed in Table 6 in a cell or tissue sample, e.g., in a cell or tissue sample obtained from a subject having or at risk of having lung cancer, or in a cell culture or cell line obtained from such a subject. In some embodiments, differential expression refers to an expression level that is statistically different in a cell or tissue obtained from a subject having cancer as compared to an expression level in a cell or tissue of the same type obtained from a subject not having cancer, or as compared to a reference level. In some embodiments, the cell or tissue is a histologically normal cell or tissue. In some embodiments, the compounds identified by the methods provided herein are anti-cancer compounds, for example, cytotoxic, cytostatic, anti-angiogenic, or anti-metastatic compounds. In some embodiments, the compounds identified by the methods provided herein are compounds that reduce the risk of developing cancer or cancer-protective compounds. In some embodiments, the method comprises screening a plurality of compounds, e.g., a compound library, for example, in order to identify one or more candidate compounds. In some embodiments, a candidate compound can be further modified or validated in subsequent studies.

In some embodiments, the method comprises providing a cell or tissue sample, e.g., obtained from or derived from a subject having lung cancer or being at an increased risk to develop lung cancer as compared to an average subject, or from a cell culture or cell line, e.g., a cell culture or cell line from a subject having lung cancer or being at an increased risk to develop lung cancer. In some embodiments, the method comprises determining that one or more informative miRNAs, e.g., miRNAs disclosed in Table 6, are differentially expressed in the cell or tissue sample, e.g., by using a method provided herein. In some embodiments the method for identifying the compound comprises contacting the cell or tissue sample differentially expressing one or more miRNAs of Table 6 with a candidate compound, and determining the level of expression of the at least one miRNA in the cell or tissue sample after the contacting. In some embodiments, if the cell ceases to express the one or more informative miRNA at a differential level after the contacting with the candidate compound, the compound is identified as a compound that reverses differential expression of one or more informative miRNAs, e.g., one or more miRNAs disclosed in Table 6. In some embodiments, a reversal of differential expression of an informative miRNA as described herein can serve as a proxy for reversal of one or more characteristics obtained by the cell or tissue, e.g. associated with carcinogenesis. In some embodiments, a reversal of differential expression of an informative miRNA as described herein can serve as a proxy for reversal of one or more clinically relevant aspects of lung cancer or for a risk of developing lung cancer. In some embodiments, a compound determined to diminish or reverse differential expression of an informative miRNA described herein, e.g., a miRNA described in Table 6, is identified as an anti-cancer compound, e.g., an anti-lung cancer compound. In some embodiments, the method further comprises determining the survival and/or proliferation of a cell or tissue sample derived from a subject having lung cancer or at an elevated risk of lung cancer before and after contacting with the candidate compound or with a compound identified to reverse differential expression of one or more informative miRNAs disclosed herein. In some embodiments, if the compound is determined to reduce the risk of carcinogenesis and/or to inhibit proliferation and/or survival, the compound is identified as an anti-cancer compound. In some embodiments, the compound can be a cytotoxic or cytostatic anti-cancer compound.

### Computer Implemented Methods

Methods disclosed herein may be implemented in any of numerous ways. For example, certain embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, some aspects of this disclosure may be embodied as a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of this disclosure discussed above. The computer readable medium or media can be transportable, such that the program or programs stored thereon can be loaded onto one or more different computers or other processors to implement various aspects of the present disclosure as discussed above. As used herein, the term “non-transitory computer-readable storage medium” encompasses only a computer-readable medium that can be considered to be a manufacture (i.e., article of manufacture) or a machine.

The terms “program” or “software” are used herein in a generic sense to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

As used herein, the term “database” generally refers to a collection of data arranged for ease and speed of search and retrieval. Further, a database typically comprises logical and physical data structures. Those skilled in the art will recognize the methods described herein may be used with any type of database including a relational database, an object-relational database and an XML-based database, where XML stands for “eXtensible-Markup-Language”. For example, the miRNA gene expression database maybe stored in and retrieved from a database. The miRNA gene expression information may be stored in or indexed in a manner that relates the gene expression information with a variety of other relevant information (e.g., information relevant for creating a report or document that aids a physician in establishing treatment protocols and/or making diagnostic determinations, or information that aids in tracking patient samples). Such relevant information may include, for example, patient identification information, ordering physician identification information, information regarding an ordering physician's office (e.g., address, telephone number), information regarding the origin of a biological sample (e.g., tissue type, date of sampling), biological sample processing information, sample quality control information, biological sample storage information, gene annotation information, lung-cancer risk classifier information, lung cancer risk factor information, payment information, order date information, etc.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

In some aspects of this disclosure, computer implemented methods for processing genomic information are provided. The methods generally involve obtaining data representing expression levels in a biological sample of one or more informative-miRNAs (e.g., at least two miRNAs selected from Table 6) and determining the likelihood that the subject has lung cancer based at least in part on the expression levels. Any of the statistical or classification methods disclosed herein may be incorporated into the computer implemented methods. In some embodiments, the methods involve calculating a risk-score indicative of the likelihood that the subject has lung cancer. Computing the risk-score may involve a determination of the combination (e.g., sum, product) of weighted expression levels, in which the expression levels are weighted by their relative contribution to predicting increased likelihood of having lung cancer. The computer implemented methods may also involve generating a report that summarizes the results of the gene expression analysis, such as by specifying the risk-score. Such methods may also involve transmitting the report to a health care provider of the subject.

### Compositions and Kits

In some aspects, compositions and related methods are provided that are useful for determining expression levels of informative-miRNAs. For example, composition are provided that consist essentially of nucleic acid probes that specifically hybridizes with informative-miRNAs or with nucleic acids having sequences complementary to informative-miRNAs. These compositions may also include probes that specifically hybridize with control miRNAs or nucleic acids complementary thereto. These compositions may also include appropriate buffers, salts or detection reagents. The nucleic acid probes may be fixed directly or indirectly to a solid support (e.g., a glass, plastic or silicon chip) or a bead (e.g., a magnetic bead). The nucleic acid probes may be customized for used in a bead-based nucleic acid detection assays.

In some embodiments, compositions are provided that comprise up to 5, up to 10, up to 25, up to 50, up to 100, or up to 200 nucleic acid probes. In some cases, each of the nucleic acid probes specifically hybridizes with an miRNA selected from Table 6 or with a nucleic acid having a sequence complementary to the miRNA. In some cases, each of at least 2, at least 3, at least 4, at least 5, at least 6, at least 7, at least 8, at least 9, at least 10, or at least 20 of the nucleic acid probes specifically hybridizes with an miRNA selected from Table 6 or with a nucleic acid having a sequence complementary to the miRNA. The compositions may be prepared for detecting different miRNAs in biochemically separate reactions, or for detecting multiple miRNA the same biochemical reactions.

Also provided herein are oligonucleotide (nucleic acid) arrays that are useful in the methods for determining levels of multiple informative-miRNAs simultaneously. Such arrays may be obtained or produced from commercial sources. Methods for producing nucleic acid arrays are also well known in the art. For example, nucleic acid arrays may be constructed by immobilizing to a solid support large numbers of oligonucleotides, polynucleotides, or cDNAs capable of hybridizing to nucleic acids corresponding to miRNAs, or portions thereof. The skilled artisan is referred to Chapter 22 “Nucleic Acid Arrays” of Current Protocols In Molecular Biology (Eds. Ausubel et al. John Wiley and #38; Sons NY, 2000) or Liu C G, et al., An oligonucleotide microchip for genome-wide microRNA profiling in human and mouse tissues. Proc Natl Acad Sci USA. 2004 Jun. 29; 101(26):9740-4, which provide non-limiting examples of methods relating to nucleic acid array construction and use in detection of nucleic acids of interest. In some embodiments, the arrays comprise, or consist essentially of, binding probes for at least 2, at least 5, at least 10, at least 20, at least 50, at least 60, at least 70 or more informative-miRNAs selected from Table 6. In some embodiments, an array comprises or consists of 1, 2, 3, 4, 5, 6, 7, 8, 9, or 10 of the miRNAs selected from Table 6. In some embodiments, an array comprises or consists of 4, 5, or 6 of the miRNAs selected from Table 6. Kits comprising the oligonucleotide arrays are also provided. Kits may include nucleic acid labeling reagents and instructions for determining expression levels using the arrays.

The compositions described herein can be provided as a kit for determining and evaluating expression levels of informative-miRNAs. The compositions may be assembled into diagnostic or research kits to facilitate their use in diagnostic or research applications. A kit may include one or more containers housing one or more of the components provided in this disclosure and instructions for use. Specifically, such kits may include one or more compositions described herein, along with instructions describing the intended application and the proper use of these compositions. Kits may contain the components in appropriate concentrations or quantities for running various experiments.

The kit may be designed to facilitate use of the methods described herein by researchers, health care providers, diagnostic laboratories, or other entities and can take many forms. Each of the compositions of the kit, where applicable, may be provided in liquid form (e.g., in solution), or in solid form, (e.g., a dry powder). In certain cases, some of the compositions may be constitutable or otherwise processable, for example, by the addition of a suitable solvent or other substance, which may or may not be provided with the kit. As used herein, “instructions” can define a component of instruction and/or promotion, and typically involve written instructions on or associated with packaging of one or more component disclosed herein. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of diagnostic or biological products, which instructions can also reflects approval by the agency.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The components may be in the form of a liquid, gel or solid (powder). The components may be prepared sterilely and shipped refrigerated. Alternatively they may be housed in a vial or other container for storage. A second container may have other components prepared sterilely.

As used herein, the terms “approximately” or “about” in reference to a number are generally taken to include numbers that fall within a range of 1%, 5%, 10%, 15%, or 20% in either direction (greater than or less than) of the number unless otherwise stated or otherwise evident from the context (except where such number would be less than 0% or exceed 100% of a possible value).

All references described herein are incorporated by reference for the purposes described herein.

Exemplary embodiments will be described in more detail by the following examples. These embodiments are exemplary, which one skilled in the art will recognize is not limited to the exemplary embodiments.

### EXAMPLES

### Introduction

Applicants have conducted a study to identify airway field of injury biomarkers using RNA recovered from bronchial epithelial cells. Histologically normal appearing cells were collected from the mainstem bronchus during bronchoscopy, using a standard bronchoscopy brush. RNA was recovered from each of the bronchial brushing samples, and was then fractionated into high and low molecular weight fractions which are then archived. The high-MW fraction were used on mRNA expression profiling analyses. The low-MW fractions were found to be enriched for miRNAs. All subjects in the study have been characterized as either having cancer, or are cancer-free (“no-cancer”). The diagnosis of cancer, in all cases, was made by pathology from cells or tissue that were obtained either through bronchoscopy, or in the cases where bronchoscopy was not successful, by follow-up procedures, such as fine-needle aspirate (FNA), surgery (e.g., thoracoscopy, thoracotomy, or mediastinoscopy), or some other technique.

Samples:

The study was designed to analyze a moderately sized set of RNA samples representing a balanced set of cancers (n=30) and no-cancers (n=30). Samples were randomly selected to result in a balance of average clinical and demographic characteristics (such as age, sex, smoking status, smoking history, and RNA quality) between the two classes. Table 1 below shows 30% current smokers for both the cancer-group as well as the no-cancer-group. Likewise smoking history (measured in pack-years; PY), median age, and % females (and males) are well matched between cancers and no-cancers.

TABLE 1
Patient Characteristics

| | | Median | | |
|---|---|---|---|---|
| | Current | Pack | Median | |
| | Smoker | Years | Age | Females |
| Cancer Group | 0.30 | 35 | 64 | 0.37 |
| No-Cancer | 0.30 | 33 | 60 | 0.33 |
| Group | | | | |

Sample Preparation:

All bronchial epithelial cells (BECs) were collected into Qiagen RNA Protect Cell to preserve the RNA for shipping and storage. Upon receipt in the lab, RNA was recovered using Qiagen miRNeasy kits, according to manufacturer's procedures. RNA was subsequently fractioned into high molecular weight (HMW)(>200 nucleotides) and low molecular weight (LMW) fractions (<200 nt) using the Qiagen kit. All RNA preps had been previously prepared and stored at −80° C. The quantity of RNA in each fraction was determined using A260/280 readings on a spectrophotometer.

Labeling & Hybridization:

The LMW fractions were labeled using the Genisphere Flashtag™ kit, and hybridized on Affymetrix Genechip miRNA arrays. Hybridizations were conducted according to manufacturer's procedures using Affymetrix hybridization ovens. Arrays were then washed and scanned using Affymetrix equipment.

Data Analysis:

Affymetrix microRNA microarrays contain probes targeting small non-coding RNA of several species, including homo sapiens (“HSA” probe). All analysis of microarray data reported here was restricted to the HSA probe. Microarray CEL files were normalized using Log 2 expression value of Robust Multi-Chip Average (RMA).

The quality of each array hybridization result was assessed using standard array-QC metrics, such as: present %, scale factor and average background for both miRNA probes and non-miRNA probes on the array. Visualization procedures such as the score plot of PCA, hierarchical clustering dendrogram and box plot were used to identify outliers.

Demonstration of differentially expressed miRNAs was performed using a t-test. Differentially expressed miRNAs were reported with a p-value≦0.05.

Selected genes were evaluated for the ability to predict cancer, based on analysis of expression levels of samples with known cancer status (either “cancer” confirmed by pathology, or “no-cancer”). Classification was performed to select using a stratified Monte-Carlo cross validation approach (also called random split) with up to 500 iterations. Results reported below were obtained using Support Vector Machines (SVM) and Linear Discriminant Analysis. Analyses were conducting using MATLAB. Predictive models were constructed using a training set, then predicted in a separate test set, and the performance [e.g., sensitivity, specificity, accuracy, and area under the curve (AUC)] was recorded. Samples were then randomized for up to 500 iterations and the performance was reported as the average over all of the iterations. In reference to the following standard confusion matrix, accuracy was defined as, (TP+TN)/(TP+FP+FN+TN), sensitivity was defined as (TP)/(TP+FN), and specificity was defined as (TN)/(FP+TN).

Condition

| | | |
|---|---|---|
| Confusion | | Negative |
| Matrix 1 | Positive (Cancer) | (No Cancer) |

| | | | |
|---|---|---|---|
| Test | Positive | True Positive | False Positive |
| Outcome | (Cancer) | (TP) | (FP) |
| | | | (Type I error) |
| | Negative | False Negative | True Negative |
| | (No | (FN) | (TN) |
| | Cancer) | (Type II Error) | |

QC of microRNA Array Data:

Principal component analysis (PCA) of all 60 microarray results was performed after RMA normalization, to analyze for outlier samples. PCA analysis (below) revealed 2 outlier samples that were subsequently removed from further analyses. The two samples (5-0054 and 13-0116, corresponded to a cancer and no-cancer, respectively).

### Results

Differentially Expressed miRNAs:

A t-test analysis comparing expression intensities between cancers and no-cancers for the 847 HSA probes was performed to determine the number of differentially expressed (DE) microRNAs. The number of DE miRNAs can be defined at different significance levels. Using p-value cut-offs, we found that 71 miRNAs were DE at a cut-off of p-value≦0.05. Higher significance cut-offs lead to smaller DE miRNAs as shown in Table 2 below.

TABLE 2
T-Test Results

| P-Value cutoff | DE probe sets |
|---|---|
| 0.05 | 71 |
| 0.01 | 23 |
| 0.005 | 14 |
| 0.001 | 3 |

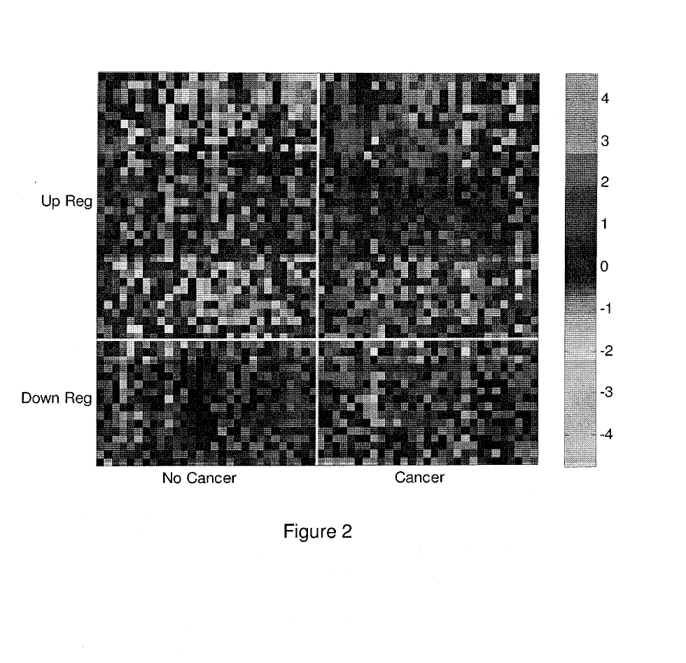

A heatmap of the top 50 most DE miRNAs comparing expression intensities for cancers and no-cancers was generated using unsupervised clustering analysis. The heatmap is separated to show miRNAs up-regulated (red) in cancer patients versus those down-regulated (green) in cancer. (See FIG. 2.)

Differentially expressed miRNAs are listed in the Tables below. In each table the microRNA name is indicated (“PS”), as well as the rank according to the t-statistic p-value, the p-value for a given microRNA probe based on differential expression between the 29 cancers and 29 no-cancers, and the probe weight. The weight was calculated as the difference in average expression intensity between cancers and no-cancers, normalized to the sum of the standard deviations. This represents a signal to noise (S/N) value of differential expression for a given microRNA in this samples set.

The probe weights were used to describe the significance of a specific probe (or gene) to differentiate cancer from control (i.e., no-cancer) patients. It was calculated as the difference in expression intensity between the two classes normalized to the sum of the standard deviation in signal intensity, and as such can be thought of as the signal-to-noise ratio. The formula is, W=(μ1−μ0)/(σ1+σ0), where μ1 is the mean signal intensity of cancer samples, μ0 is the mean intensity of no-cancer samples, and σ1 and σ0 correspond to the standard deviations of the cancer and no-cancer intensities, respectively.

Table 3 lists the microRNAs found to be differentially expressed in cancer patients consistent. These results indicate that the airway field of injury concept is applicable to miRNA expression. All miRNAs are determined based on a t-statistic (p≦0.05) of differential expression.

A subset of the total sample set were found to include patients with a history of other cancers. A separate analysis was conducted to determine the differentially expressed microRNAs after excluding these patients. This list, shown in Table 4, contains 48 miRNAs. It was observed that 37 of the 48 miRNAs in Table 4 match those in Table 3 suggesting that the biological mechanism is similar for both sample sets. Assessment of prediction accuracy is described below.

TABLE 3
71 microRNAs differentially expressed at a p-value ≦0.05 for all
cancer and no-cancer samples (sample set 1).

| Affy Probe Set | Rank | P-value | Weights |
|---|---|---|---|
| ‘hsa-miR-210_st’ | 1 | 6.87E−05 | 0.568975 |
| ‘hsa-miR-378_st’ | 2 | 0.000145 | 0.536297 |
| ‘hsa-miR-221-star_st’ | 3 | 0.000177 | −0.52761 |
| ‘hsa-miR-320b_st’ | 4 | 0.001054 | 0.458915 |
| ‘hsa-miR-1226-star_st’ | 5 | 0.001241 | −0.44697 |
| ‘hsa-miR-744_st’ | 6 | 0.001313 | 0.445062 |
| ‘hsa-miR-320a_st’ | 7 | 0.001324 | 0.448965 |
| ‘hsa-miR-1243_st’ | 8 | 0.002324 | −0.43116 |
| ‘hsa-miR-345_st’ | 9 | 0.002642 | 0.416316 |
| ‘hsa-miR-200b_st’ | 10 | 0.003198 | −0.40761 |
| ‘hsa-miR-574-3p_st’ | 11 | 0.003684 | 0.398697 |
| ‘hsa-miR-361-5p_st’ | 12 | 0.00388 | 0.401963 |
| ‘hsa-miR-423-5p_st’ | 13 | 0.004491 | 0.389096 |
| ‘hsa-miR-99b-star_st’ | 14 | 0.004903 | 0.385633 |
| ‘hsa-let-7b_st’ | 15 | 0.005046 | 0.388982 |
| ‘hsa-miR-574-5p_st’ | 16 | 0.005147 | 0.38552 |
| ‘hsa-miR-1180_st’ | 17 | 0.0055 | 0.383159 |
| ‘hsa-miR-423-3p_st’ | 18 | 0.006119 | 0.374136 |
| ‘hsa-miR-1307_st’ | 19 | 0.006153 | 0.377734 |
| ‘hsa-miR-320c_st’ | 20 | 0.006159 | 0.377882 |
| ‘hsa-miR-342-3p_st’ | 21 | 0.006906 | 0.372296 |
| ‘hsa-miR-520d-5p_st’ | 22 | 0.007097 | −0.38172 |
| ‘hsa-miR-99b_st’ | 23 | 0.009429 | 0.35545 |
| ‘hsa-miR-500-star_st’ | 24 | 0.010659 | 0.360776 |
| ‘hsa-miR-363_st’ | 25 | 0.010885 | −0.34611 |
| ‘hsa-miR-491-5p_st’ | 26 | 0.011519 | 0.344197 |
| ‘hsa-miR-339-3p_st’ | 27 | 0.011839 | 0.342006 |
| ‘hsa-miR-29a_st’ | 28 | 0.011896 | −0.34306 |
| ‘hsa-miR-652_st’ | 29 | 0.01401 | 0.33312 |
| ‘hsa-miR-376c_st’ | 30 | 0.01497 | −0.33001 |
| ‘hsa-miR-140-3p_st’ | 31 | 0.015196 | 0.331547 |
| ‘hsa-miR-324-5p_st’ | 32 | 0.015424 | 0.328541 |
| ‘hsa-miR-34c-3p_st’ | 33 | 0.015507 | 0.345525 |
| ‘hsa-miR-146a_st’ | 34 | 0.015712 | −0.32762 |
| ‘hsa-miR-92b_st’ | 35 | 0.01599 | 0.330939 |
| ‘hsa-miR-523_st’ | 36 | 0.017787 | 0.320898 |
| ‘hsa-miR-454-star_st’ | 37 | 0.020692 | 0.313561 |
| ‘hsa-miR-1208_st’ | 38 | 0.020822 | −0.32387 |
| ‘hsa-miR-769-5p_st’ | 39 | 0.022835 | 0.30835 |
| ‘hsa-miR-193a-3p_st’ | 40 | 0.024826 | −0.30296 |
| ‘hsa-let-7i_st’ | 41 | 0.025217 | −0.30626 |
| ‘hsa-miR-1249_st’ | 42 | 0.025937 | 0.303357 |
| ‘hsa-miR-302f_st’ | 43 | 0.026681 | 0.299001 |
| ‘hsa-miR-1233_st’ | 44 | 0.027396 | 0.300125 |
| ‘hsa-miR-874_st’ | 45 | 0.027746 | 0.297709 |
| ‘hsa-miR-889_st’ | 46 | 0.028506 | −0.29531 |
| ‘hsa-miR-199b-5p_st’ | 47 | 0.029967 | −0.29865 |
| ‘hsa-miR-224_st’ | 48 | 0.032271 | −0.29057 |
| ‘hsa-miR-221_st’ | 49 | 0.032379 | −0.29333 |
| ‘hsa-miR-143-star_st’ | 50 | 0.032652 | 0.29316 |
| ‘hsa-miR-301a_st’ | 51 | 0.035429 | −0.28509 |
| ‘hsa-miR-1224-5p_st’ | 52 | 0.036693 | −0.28185 |
| ‘hsa-miR-568_st’ | 53 | 0.038112 | 0.284281 |
| ‘hsa-miR-502-3p_st’ | 54 | 0.038243 | 0.279271 |
| ‘hsa-miR-191-star_st’ | 55 | 0.038626 | 0.280226 |
| ‘hsa-miR-633_st’ | 56 | 0.03929 | 0.277467 |
| ‘hsa-miR-455-5p_st’ | 57 | 0.039654 | −0.28317 |
| ‘hsa-miR-612_st’ | 58 | 0.040218 | 0.275801 |
| ‘hsa-miR-519b-5p_st’ | 59 | 0.040316 | −0.28276 |
| ‘hsa-miR-23b-star_st’ | 60 | 0.041151 | 0.275678 |
| ‘hsa-miR-339-5p_st’ | 61 | 0.041414 | 0.275004 |
| ‘hsa-miR-191_st’ | 62 | 0.042209 | 0.273061 |
| ‘hsa-miR-141-star_st’ | 63 | 0.045675 | −0.2783 |
| ‘hsa-miR-1247_st’ | 64 | 0.046341 | 0.268726 |
| ‘hsa-miR-324-3p_st’ | 65 | 0.046408 | 0.268704 |
| ‘hsa-miR-200b-star_st’ | 66 | 0.046417 | 0.268073 |
| ‘hsa-miR-187_st’ | 67 | 0.046988 | 0.267837 |
| ‘hsa-miR-19a_st’ | 68 | 0.048182 | −0.26879 |
| ‘hsa-miR-130a-star_st’ | 69 | 0.04897 | 0.265108 |
| ‘hsa-miR-374a_st’ | 70 | 0.049212 | −0.26509 |
| ‘hsa-miR-1275_st’ | 71 | 0.049393 | 0.264329 |

TABLE 4
48 microRNAs differentially expressed at a p-value ≦0.05 for Sample
set 2 (cancer and no-cancer patients, exclusive of patients with a
personal history of other cancers).

| Affy Probe Set | Rank | P-value | Weights |
|---|---|---|---|
| ‘hsa-miR-221-star_st’ | 1 | 0.000395 | −0.55941 |
| ‘hsa-miR-146a_st’ | 2 | 0.001317 | −0.50082 |
| ‘hsa-miR-378_st’ | 3 | 0.00156 | 0.492075 |
| ‘hsa-miR-210_st’ | 4 | 0.001639 | 0.492438 |
| ‘hsa-miR-1243_st’ | 5 | 0.003329 | −0.45923 |
| ‘hsa-miR-1226-star_st’ | 6 | 0.004129 | −0.44108 |
| ‘hsa-miR-320b_st’ | 7 | 0.004405 | 0.440703 |
| ‘hsa-miR-320a_st’ | 8 | 0.005718 | 0.426641 |
| ‘hsa-miR-92b_st’ | 9 | 0.007544 | 0.410531 |
| ‘hsa-miR-376c_st’ | 10 | 0.013065 | −0.37701 |
| ‘hsa-miR-320c_st’ | 11 | 0.013342 | 0.379313 |
| ‘hsa-miR-1180_st’ | 12 | 0.014445 | 0.375373 |
| ‘hsa-miR-200b_st’ | 13 | 0.015287 | −0.37085 |
| ‘hsa-miR-363_st’ | 14 | 0.015983 | −0.36533 |
| ‘hsa-miR-1307_st’ | 15 | 0.017434 | 0.363009 |
| ‘hsa-miR-1233_st’ | 16 | 0.017781 | 0.365248 |
| ‘hsa-miR-744_st’ | 17 | 0.018857 | 0.355406 |
| ‘hsa-miR-199b-5p_st’ | 18 | 0.018917 | −0.36017 |
| ‘hsa-miR-99b-star_st’ | 19 | 0.019906 | 0.353568 |
| ‘hsa-miR-517a_st’ | 20 | 0.020325 | 0.354044 |
| ‘hsa-miR-520d-5p_st’ | 21 | 0.022541 | −0.3647 |
| ‘hsa-let-7g_st’ | 22 | 0.023919 | −0.34142 |
| ‘hsa-miR-345_st’ | 23 | 0.025563 | 0.336927 |
| ‘hsa-miR-374a_st’ | 24 | 0.02575 | −0.33752 |
| ‘hsa-miR-574-3p_st’ | 25 | 0.026295 | 0.33546 |
| ‘hsa-miR-936_st’ | 26 | 0.02747 | −0.33312 |
| ‘hsa-miR-25_st’ | 27 | 0.027636 | −0.33205 |
| ‘hsa-miR-130a-star_st’ | 28 | 0.027641 | 0.332345 |
| ‘hsa-let-7b_st’ | 29 | 0.028523 | 0.332767 |
| ‘hsa-miR-412_st’ | 30 | 0.029773 | −0.32819 |
| ‘hsa-miR-1225-3p_st’ | 31 | 0.030978 | 0.338824 |
| ‘hsa-miR-491-5p_st’ | 32 | 0.033055 | 0.32226 |
| ‘hsa-miR-454-star_st’ | 33 | 0.033943 | 0.319842 |
| ‘hsa-miR-500-star_st’ | 34 | 0.034007 | 0.31986 |
| ‘hsa-miR-633_st’ | 35 | 0.03446 | 0.320799 |
| ‘hsa-miR-301a_st’ | 36 | 0.035419 | −0.31783 |
| ‘hsa-miR-374b_st’ | 37 | 0.038302 | −0.32024 |
| ‘hsa-miR-99b_st’ | 38 | 0.039904 | 0.310847 |
| ‘hsa-miR-423-5p_st’ | 39 | 0.040258 | 0.309065 |
| ‘hsa-miR-568_st’ | 40 | 0.040911 | 0.32216 |
| ‘hsa-miR-519b-5p_st’ | 41 | 0.040925 | −0.3234 |
| ‘hsa-miR-34c-3p_st’ | 42 | 0.041785 | 0.324648 |
| ‘hsa-miR-483-5p_st’ | 43 | 0.042064 | −0.33619 |
| ‘hsa-miR-224_st’ | 44 | 0.042698 | −0.30643 |
| ‘hsa-miR-1226_st’ | 45 | 0.043454 | −0.30422 |
| ‘hsa-miR-30e-star_st’ | 46 | 0.046102 | −0.30201 |
| ‘hsa-miR-423-3p_st’ | 47 | 0.046736 | 0.299029 |
| ‘hsa-miR-378-star_st’ | 48 | 0.049595 | 0.294346 |

TABLE 5
37 microRNAs differentially expressed at a p-value ≦0.05 for both
sample sets 1 and 2

| | Sample Set 1 | | Sample Set 2 | |
|---|---|---|---|---|
| Affy Probe Sets | P-value | Weights | P-value | Weights |
| ‘hsa-miR-210_st’ | 0.0000687 | 0.56898 | 0.00164 | 0.49244 |
| ‘hsa-miR-378_st’ | 0.000145 | 0.5363 | 0.00156 | 0.49208 |
| ‘hsa-miR-221-star_st’ | 0.000177 | −0.5276 | 0.0004 | −0.55941 |
| ‘hsa-miR-320b_st’ | 0.001054 | 0.45892 | 0.00441 | 0.4407 |
| ‘hsa-miR-1226-star_st’ | 0.001241 | −0.447 | 0.00413 | −0.44108 |
| ‘hsa-miR-744_st’ | 0.001313 | 0.44506 | 0.01886 | 0.35541 |
| ‘hsa-miR-320a_st’ | 0.001324 | 0.44897 | 0.00572 | 0.42664 |
| ‘hsa-miR-1243_st’ | 0.002324 | −0.4312 | 0.00333 | −0.45923 |
| ‘hsa-miR-345_st’ | 0.002642 | 0.41632 | 0.02556 | 0.33693 |
| ‘hsa-miR-200b_st’ | 0.003198 | −0.4076 | 0.01529 | −0.37085 |
| ‘hsa-miR-574-3p_st’ | 0.003684 | 0.3987 | 0.0263 | 0.33546 |
| ‘hsa-miR-423-5p_st’ | 0.004491 | 0.3891 | 0.04026 | 0.30907 |
| ‘hsa-miR-99b-star_st’ | 0.004903 | 0.38563 | 0.01991 | 0.35357 |
| ‘hsa-let-7b_st’ | 0.005046 | 0.38898 | 0.02852 | 0.33277 |
| ‘hsa-miR-1180_st’ | 0.0055 | 0.38316 | 0.01445 | 0.37537 |
| ‘hsa-miR-423-3p_st’ | 0.006119 | 0.37414 | 0.04674 | 0.29903 |
| ‘hsa-miR-1307_st’ | 0.006153 | 0.37773 | 0.01743 | 0.36301 |
| ‘hsa-miR-320c_st’ | 0.006159 | 0.37788 | 0.01334 | 0.37931 |
| ‘hsa-miR-520d-5p_st’ | 0.007097 | −0.3817 | 0.02254 | −0.3647 |
| ‘hsa-miR-99b_st’ | 0.009429 | 0.35545 | 0.0399 | 0.31085 |
| ‘hsa-miR-500-star_st’ | 0.010659 | 0.36078 | 0.03401 | 0.31986 |
| ‘hsa-miR-363_st’ | 0.010885 | −0.3461 | 0.01598 | −0.36533 |
| ‘hsa-miR-491-5p_st’ | 0.011519 | 0.3442 | 0.03306 | 0.32226 |
| ‘hsa-miR-376c_st’ | 0.01497 | −0.33 | 0.01307 | −0.37701 |
| ‘hsa-miR-34c-3p_st’ | 0.015507 | 0.34553 | 0.04179 | 0.32465 |
| ‘hsa-miR-146a_st’ | 0.015712 | −0.3276 | 0.00132 | −0.50082 |
| ‘hsa-miR-92b_st’ | 0.01599 | 0.33094 | 0.00754 | 0.41053 |
| ‘hsa-miR-454-star_st’ | 0.020692 | 0.31356 | 0.03394 | 0.31984 |
| ‘hsa-miR-1233_st’ | 0.027396 | 0.30013 | 0.01778 | 0.36525 |
| ‘hsa-miR-199b-5p_st’ | 0.029967 | −0.2987 | 0.01892 | −0.36017 |
| ‘hsa-miR-224_st’ | 0.032271 | −0.2906 | 0.0427 | −0.30643 |
| ‘hsa-miR-301a_st’ | 0.035429 | −0.2851 | 0.03542 | −0.31783 |
| ‘hsa-miR-568_st’ | 0.038112 | 0.28428 | 0.04091 | 0.32216 |
| ‘hsa-miR-633_st’ | 0.03929 | 0.27747 | 0.03446 | 0.3208 |
| ‘hsa-miR-519b-5p_st’ | 0.040316 | −0.2828 | 0.04093 | −0.3234 |
| ‘hsa-miR-130a-star_st’ | 0.04897 | 0.26511 | 0.02764 | 0.33235 |
| ‘hsa-miR-374a_st’ | 0.049212 | −0.2651 | 0.02575 | −0.33752 |

TABLE 6
Informative-miRNAs

| | SEQ | | Accession |
|---|---|---|---|
| | ID | Mature miRNA | Number- |
| Gene ID | NO: | Sequence | miRBase |
| hsa-let-7b | 1 | UGAGGUAGUAGGU | MIMAT0000063 |
| | | UGUGUGGUU | |
| hsa-let-7g | 2 | UGAGGUAGUAGUU | MIMAT0000414 |
| | | UGUACAGUU | |
| hsa-let-7i | 3 | UGAGGUAGUAGUU | MIMAT0000415 |
| | | UGUGCUGUU | |
| hsa-miR-1180 | 4 | UUUCCGGCUCGCG | MIMAT0005825 |
| | | UGGGUGUGU | |
| hsa-miR-1208 | 5 | UCACUGUUCAGAC | MIMAT0005873 |
| | | AGGCGGA | |
| hsa-miR-1224- | 6 | GUGAGGACUCGGG | MIMAT0005458 |
| 5p | | AGGUGG | |
| hsa-miR-1225- | 7 | UGAGCCCCUGUGC | MIMAT0005573 |
| 3p | | CGCCCCCAG | |
| hsa-miR-1226 | 8 | UCACCAGCCCUGU | MIMAT0005577 |
| | | GUUCCCUAG | |
| hsa-miR-1226* | 9 | GUGAGGGCAUGCA | MIMAT0005576 |
| | | GGCCUGGAUGGGG | |
| hsa-miR-1233 | 10 | UGAGCCCUGUCCU | MIMAT0005588 |
| | | CCCGCAG | |
| hsa-miR-1243 | 11 | AACUGGAUCAAUU | MIMAT0005894 |
| | | AUAGGAGUG | |
| hsa-miR-1247 | 12 | ACCCGUCCCGUUC | MIMAT0005899 |
| | | GUCCCCGGA | |
| hsa-miR-1249 | 13 | ACGCCCUUCCCCC | MIMAT0005901 |
| | | CCUUCUUCA | |
| hsa-miR-1275 | 14 | GUGGGGGAGAGGC | MIMAT0005929 |
| | | UGUC | |
| hsa-miR-1307 | 15 | ACUCGGCGUGGCG | MIMAT0005951 |
| | | UCGGUCGUG | |
| hsa-miR-130a* | 16 | UUCACAUUGUGCU | MIMAT0004593 |
| | | ACUGUCUGC | |
| hsa-miR-140-3p | 17 | UACCACAGGGUAG | MIMAT0004597 |
| | | AACCACGG | |
| hsa-miR-141* | 18 | CAUCUUCCAGUAC | MIMAT0004598 |
| | | AGUGUUGGA | |
| hsa-miR-143* | 19 | GGUGCAGUGCUGC | MIMAT0004599 |
| | | AUCUCUGGU | |
| hsa-miR-146a | 20 | UGAGAACUGAAUU | MIMAT0000449 |
| | | CCAUGGGUU | |
| hsa-miR-187 | 21 | UCGUGUCUUGUGU | MIMAT0000262 |
| | | UGCAGCCGG | |
| hsa-miR-191 | 22 | CAACGGAAUCCCA | MIMAT0000440 |
| | | AAAGCAGCUG | |
| hsa-miR-191* | 23 | GCUGCGCUUGGAU | MIMAT0001618 |
| | | UUCGUCCCC | |
| hsa-miR-193a- | 24 | AACUGGCCUACAA | MIMAT0000459 |
| 3p | | AGUCCCAGU | |
| hsa-miR-199b- | 25 | CCCAGUGUUUAGA | MIMAT0000263 |
| 5p | | CUAUCUGUUC | |
| hsa-miR-19a | 26 | UGUGCAAAUCUAU | MIMAT0000073 |
| | | GCAAAACUGA | |
| hsa-miR-200b | 27 | UAAUACUGCCUGG | MIMAT0000318 |
| | | UAAUGAUGA | |
| hsa-miR-200b* | 28 | CAUCUUACUGGGC | MIMAT0004571 |
| | | AGCAUUGGA | |
| hsa-miR-210 | 29 | CUGUGCGUGUGAC | MIMAT0000267 |
| | | AGCGGCUGA | |
| hsa-miR-221 | 30 | AGCUACAUUGUCU | MIMAT0000278 |
| | | GCUGGGUUUC | |
| hsa-miR-221* | 31 | ACCUGGCAUACAA | MIMAT0004568 |
| | | UGUAGAUUU | |
| hsa-miR-224 | 32 | CAAGUCACUAGUG | MIMAT0000281 |
| | | GUUCCGUU | |
| hsa-miR-23b* | 33 | UGGGUUCCUGGCA | MIMAT0004587 |
| | | UGCUGAUUU | |
| hsa-miR-25 | 34 | CAUUGCACUUGUC | MIMAT0000081 |
| | | UCGGUCUGA | |
| hsa-miR-29a | 35 | UAGCACCAUCUGA | MIMAT0000086 |
| | | AAUCGGUUA | |
| hsa-miR-301a | 36 | CAGUGCAAUAGUA | MIMAT0000688 |
| | | UUGUCAAAGC | |
| hsa-miR-302f | 37 | UAAUUGCUUCCAU | MIMAT0005932 |
| | | GUUU | |
| hsa-miR-30e* | 38 | CUUUCAGUCGGAU | MIMAT0000693 |
| | | GUUUACAGC | |
| hsa-miR-320a | 39 | AAAAGCUGGGUUG | MIMAT0000510 |
| | | AGAGGGCGA | |
| hsa-miR-320b | 40 | AAAAGCUGGGUUG | MIMAT0005792 |
| | | AGAGGGCAA | |
| hsa-miR-320c | 41 | AAAAGCUGGGUUG | MIMAT0005793 |
| | | AGAGGGU | |
| hsa-miR-324-3p | 42 | ACUGCCCCAGGUG | MIMAT0000762 |
| | | CUGCUGG | |
| hsa-miR-324-5p | 43 | CGCAUCCCCUAGG | MIMAT0000761 |
| | | GCAUUGGUGU | |
| hsa-miR-339-3p | 44 | UGAGCGCCUCGAC | MIMAT0004702 |
| | | GACAGAGCCG | |
| hsa-miR-339-5p | 45 | UCCCUGUCCUCCA | MIMAT0000764 |
| | | GGAGCUCACG | |
| hsa-miR-342-3p | 46 | UCUCACACAGAAA | MIMAT0000753 |
| | | UCGCACCCGU | |
| hsa-miR-345 | 47 | GCUGACUCCUAGU | MIMAT0000772 |
| | | CCAGGGCUC | |
| hsa-miR-34c-3p | 48 | AAUCACUAACCAC | MIMAT0004677 |
| | | ACGGCCAGG | |
| hsa-miR-361-5p | 49 | UUAUCAGAAUCUC | MIMAT0000703 |
| | | CAGGGGUAC | |
| hsa-miR-363 | 50 | AAUUGCACGGUAU | MIMAT0000707 |
| | | CCAUCUGUA | |
| hsa-miR-374a | 51 | UUAUAAUACAACC | MIMAT0000727 |
| | | UGAUAAGUG | |
| hsa-miR-374b | 52 | AUAUAAUACAACC | MIMAT0004955 |
| | | UGCUAAGUG | |
| hsa-miR-376c | 53 | AACAUAGAGGAAA | MIMAT0000720 |
| | | UUCCACGU | |
| hsa-miR-378 | 54 | ACUGGACUUGGAG | MIMAT0000732 |
| | | UCAGAAGG | |
| hsa-miR-378* | 55 | CUCCUGACUCCAG | MIMAT0000731 |
| | | GUCCUGUGU | |
| hsa-miR-412 | 56 | ACUUCACCUGGUC | MIMAT0002170 |
| | | CACUAGCCGU | |
| hsa-miR-423-3p | 57 | AGCUCGGUCUGAG | MIMAT0001340 |
| | | GCCCCUCAGU | |
| hsa-miR-423-5p | 58 | UGAGGGGCAGAGA | MIMAT0004748 |
| | | GCGAGACUUU | |
| hsa-miR-454* | 59 | ACCCUAUCAAUAU | MIMAT0003884 |
| | | UGUCUCUGC | |
| hsa-miR-455-5p | 60 | UAUGUGCCUUUGG | MIMAT0003150 |
| | | ACUACAUCG | |
| hsa-miR-483-5p | 61 | AAGACGGGAGGAA | MIMAT0004761 |
| | | AGAAGGGAG | |
| hsa-miR-491-5p | 62 | AGUGGGGAACCCU | MIMAT0002807 |
| | | UCCAUGAGG | |
| hsa-miR-500* | 63 | AUGCACCUGGGCA | MIMAT0002871 |
| | | AGGAUUCUG | |
| hsa-miR-502-3p | 64 | AAUGCACCUGGGC | MIMAT0004775 |
| | | AAGGAUUCA | |
| hsa-miR-517a | 65 | AUCGUGCAUCCCU | MIMAT0002852 |
| | | UUAGAGUGU | |
| hsa-miR-519b- | 66 | CUCUAGAGGGAAG | MIMAT0005454 |
| 5p | | CGCUUUCUG | |
| hsa-miR-520d- | 67 | CUACAAAGGGAAG | MIMAT0002855 |
| 5p | | CCCUUUC | |
| hsa-miR-523 | 68 | GAACGCGCUUCCC | MIMAT0002840 |
| | | UAUAGAGGGU | |
| hsa-miR-568 | 69 | AUGUAUAAAUGUA | MIMAT0003232 |
| | | UACACAC | |
| hsa-miR-574-3p | 70 | CACGCUCAUGCAC | MIMAT0003239 |
| | | ACACCCACA | |
| hsa-miR-574-5p | 71 | UGAGUGUGUGUGU | MIMAT0004795 |
| | | GUGAGUGUGU | |
| hsa-miR-612 | 72 | GCUGGGCAGGGCU | MIMAT0003280 |
| | | UCUGAGCUCCUU | |
| hsa-miR-633 | 73 | CUAAUAGUAUCUA | MIMAT0003303 |
| | | CCACAAUAAA | |
| hsa-miR-652 | 74 | AAUGGCGCCACUA | MIMAT0003322 |
| | | GGGUUGUG | |
| hsa-miR-744 | 75 | UGCGGGGCUAGGG | MIMAT0004945 |
| | | CUAACAGCA | |
| hsa-miR-769-5p | 76 | UGAGACCUCUGGG | MIMAT0003886 |
| | | UUCUGAGCU | |
| hsa-miR-874 | 77 | CUGCCCUGGCCCG | MIMAT0004911 |
| | | AGGGACCGA | |
| hsa-miR-889 | 78 | UUAAUAUCGGACA | MIMAT0004921 |
| | | ACCAUUGU | |
| hsa-miR-92b | 79 | UAUUGCACUCGUC | MIMAT0003218 |
| | | CCGGCCUCC | |
| hsa-miR-936 | 80 | ACAGUAGAGGGAG | MIMAT0004979 |
| | | GAAUCGCAG | |
| hsa-miR-99b | 81 | CACCCGUAGAACC | MIMAT0000689 |
| | | GACCUUGCG | |
| hsa-miR-99b* | 82 | CAAGCUCGUGUCU | MIMAT0004678 |
| | | GUGGGUCCG | |

Only one strand of the respective miRNA is described. Those of skill in the art will be able to determine the complementary strand of each miRNA provided herein from the information disclosed. In some embodiments, reference to a nucleic acid molecule, e.g., a nucleic acid probe, that binds or hybridizes to a given miRNA, includes nucleic acid molecules that bind to the provided nucleic acid sequence of the respective miRNA. In some embodiments, such reference includes nucleic acid molecules that bind to a nucleic acid molecule complementary to the respective miRNA nucleic acid sequence provided.

Prediction Accuracy:

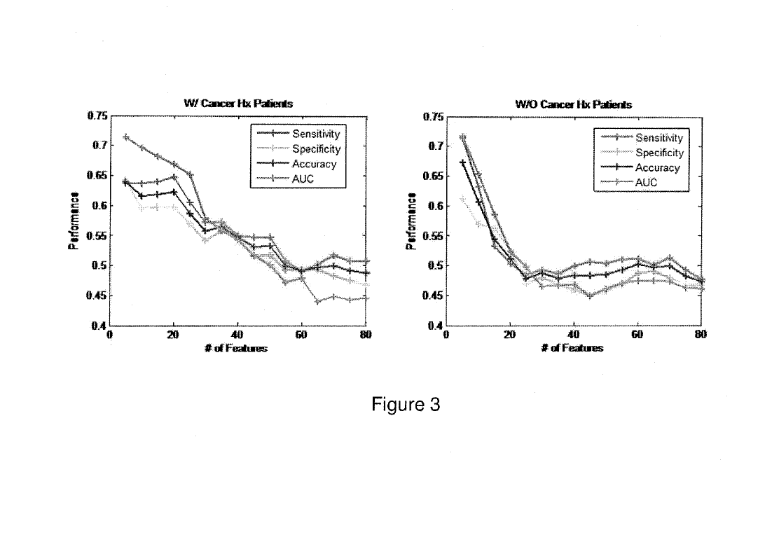

A Monte-Carlo cross-validation approach was used to assign samples to separate training and test sets, where by the accuracy of the prediction model was recorded (e.g., sensitivity, specificity, accuracy, and area under the curve (AUC) of a receiver operator characteristic (ROC) curve). The total samples set was then randomized and a second assignment into training and test sets was performed to again record prediction accuracy. This process was repeated a total of 500 times and the averaged test performance metrics were calculated across all iterations. The results are presented in FIG. 3 for two cases. The first case shows prediction accuracy for a miRNA biomarker trained using all cancers and no-cancers. In this case. it was determined that good performance can be achieved using the top 5 microRNAs. In this case, the overall accuracy was approximately 65%, with similar sensitivity and specificity. For the case in which patients with a history of cancer are removed from the analysis, the biomarker accuracy was only marginally improved. Here again, good performance was found by combining the top 5 miRNAs, in which case sensitivity is 72%, specificity is 62%, and overall accuracy is 67%. (See FIG. 3.)

Top miRNAs for sample set 1 and sample set 2 are shown in Table 7, below. Sample set 1 is defined as the case of all cancers and no-cancers (29 v 29), and sample set 2 is restricted to a subset of cancer and no-cancer subjects, exclusive of subjects with a personal history of other cancers. Note that 3 of the 5 miRNAs are common to both sample set rankings, and are found to have comparable weights.

TABLE 7
miRNAs determined as the top ranked according to sample set 1 and
sample set 2.

| Ranked according | | | Ranked according | | |
|---|---|---|---|---|---|
| to Sample set 1: | | | to Sample set 2: | | |
| miRNA ID | Rank | Weights | miRNA ID | Rank | Weights |
| hsa-miR- | 1 | 0.569 | hsa-miR-221- | 1 | −0.559 |
| 210_st | | | star_st | | |
| hsa-miR- | 2 | 0.536 | hsa-miR-146a_st | 2 | −0.501 |
| 378_st | | | | | |
| hsa-miR-221- | 3 | −0.528 | hsa-miR-378_st | 3 | 0.492 |
| star_st | | | | | |
| hsa-miR- | 4 | 0.459 | hsa-miR-210_st | 4 | 0.492 |
| 320b_st | | | | | |
| hsa-miR-1226- | 5 | −0.447 | hsa-miR-1243_st | 5 | −0.459 |
| star_st | | | | | |

### Linear Discriminant Analysis

BronchoGen (BG) scores (lung cancer-risk-scores) were determined from a linear discriminant analysis (LDA) based on the complete set of expression value. This values are determined based on a weight regression function derived from the LDA in which average expression values for hsa-miR-210, hsa-miR-378, hsa-miR-221*, hsa-miR-320b, and hsa-miR-1226* serves as regressors.

The 5 features (miRNAs) were selected according to p-value ranking of a t-test between cancers and controls. A confusion matrix based on a Monte-Carlo cross-validation analysis with ˜500 iterations was performed, in which average BronchoGen score for each sample was determined across the 500 iterations.

| Confusion | | |
|---|---|---|
| Matrix 2 | NC | C |
| Predicted NC | 23.0 | 7.0 |
| Predicted C | 7.0 | 23.0 |

According to the BG-scoring as described and the data in Table 8 below, the overall biomarker performance is summarized as follows:

Sensitivity=77%

Specificity=77%

Accuracy=77%

The BG scores are interpreted as follows, based on the biomarker training:

BG>0, predicted as cancer; and

BG<0, predicted as no-cancer.

TABLE 8
BCG Scores and Histological Findings

| Barcode | Cancer | CellType | BGScore |
|---|---|---|---|
| 1-5-0032-2.CEL | 1 | adeno | 0.806 |
| 1-1-0018-2.CEL | 1 | squamous | 0.7655 |
| 1-1-0013-2.CEL | 1 | adeno | 0.6974 |
| 1-13-0127-2.CEL | 1 | squamous | 0.602 |
| 1-13-0132-2.CEL | 1 | adeno | 0.5982 |
| 1-14-0035-2.CEL | 1 | squamous | 0.5942 |
| 1-3-0029-2.CEL | 1 | adeno | 0.5613 |
| 1-1-0014-3.CEL | 1 | adeno | 0.5468 |
| 1-14-0018-2.CEL | 1 | sclc | 0.5323 |
| 1-5-0031-2.CEL | 1 | sclc | 0.4721 |
| 1-1-0022-2.CEL | 1 | nsclc | 0.4423 |
| 1-05-0054-2.CEL | 1 | nsclc | 0.4002 |
| 1-05-0059-2.CEL | 1 | large | 0.3947 |
| 1-05-0045-2.CEL | 1 | nsclc | 0.3852 |
| 1-12-0029-2.CEL | 1 | adeno | 0.3778 |
| 1-01-0077-2.CEL | 1 | squamous | 0.2893 |
| 1-1-0032-2.CEL | 1 | sclc | 0.2883 |
| 1-7-0007-2.CEL | 1 | squamous | 0.2787 |
| 1-3-0004-2.CEL | 1 | squamous | 0.1993 |
| 1-14-0029-2.CEL | 1 | sclc | 0.1704 |
| 1-1-0025-2.CEL | 1 | sclc | 0.1501 |
| 1-1-0035-2.CEL | 1 | sclc | 0.1044 |
| 1-2-0006-2.CEL | 1 | squamous | 0.0883 |
| 1-7-0005-2.CEL | 1 | sclc | −0.1098 |
| 1-12-0011-2.CEL | 1 | adeno | −0.2148 |
| 1-5-0016-2.CEL | 1 | squamous | −0.2442 |
| 1-01-0044-2.CEL | 1 | squamous | −0.308 |
| 1-13-0135-2.CEL | 1 | sclc | −0.432 |
| 1-1-0012-2.CEL | 1 | sclc | −0.5258 |
| 1-1-0019-2.CEL | 1 | sclc | −0.622 |
| 1-3-0032-2.CEL | 0 | NC | 0.5844 |
| 1-01-0068-2.CEL | 0 | NC | 0.473 |
| 1-11-0008-2.CEL | 0 | NC | 0.4007 |
| 1-05-0077-2.CEL | 0 | NC | 0.3918 |
| 1-5-0038-2.CEL | 0 | NC | 0.3052 |
| 1-12-0005-2.CEL | 0 | NC | 0.2814 |
| 1-7-0009-2.CEL | 0 | NC | 0.0575 |
| 1-2-0023-2.CEL | 0 | NC | −0.0232 |
| 1-05-0051-2.CEL | 0 | NC | −0.0915 |
| 1-1-0008-2.CEL | 0 | NC | −0.1385 |
| 1-13-0136-2.CEL | 0 | NC | −0.1792 |
| 1-13-0149-2.CEL | 0 | NC | −0.2048 |
| 1-13-0140-2.CEL | 0 | NC | −0.2333 |
| 1-01-0089-2.CEL | 0 | NC | −0.2877 |
| 1-01-0112-2.CEL | 0 | NC | −0.3004 |
| 1-5-0035-2.CEL | 0 | NC | −0.3159 |
| 1-7-0017-2.CEL | 0 | NC | −0.3628 |
| 1-7-0015-2.CEL | 0 | NC | −0.3652 |
| 1-3-0025-2.CEL | 0 | NC | −0.4611 |
| 1-1-0038-2.CEL | 0 | NC | −0.5031 |
| 1-11-0005-2.CEL | 0 | NC | −0.5143 |
| 1-13-0119-2.CEL | 0 | NC | −0.5165 |
| 1-01-0099-2.CEL | 0 | NC | −0.594 |
| 1-5-0018-2.CEL | 0 | NC | −0.6192 |
| 1-01-0054-2.CEL | 0 | NC | −0.6292 |
| 1-05-0090-2.CEL | 0 | NC | −0.6494 |
| 1-01-0136-2.CEL | 0 | NC | −0.6939 |
| 1-9-0011-2.CEL | 0 | NC | −0.7811 |
| 1-12-0043-2.CEL | 0 | NC | −0.8078 |
| 1-13-0116-2.CEL | 0 | NC | −0.8249 |

Cancer status is indicated as either 1=cancer, or 0=no-cancer. Histological subtype, which comes from confirmed pathology of malignant cells collected is assigned as one of the following categories:
“adeno”=adenocarcinoma
“squamous”=squamous cell carcinoma
“sclc”=small cell cancer
“nsclc”=non-small cell cancer

Adeno and squamous are well-recognized NSCLC cancers. Samples labeled as “nsclc” could not be further characterized either due to poor tumor differentiation, mixed subtype, or some other factor.

Having thus described several aspects of at least one embodiment of this disclosure, it is to be appreciated that various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only and some embodiments of the disclosure are described in detail by the claims that follow.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of determining the likelihood that a subject has lung cancer, the method comprising:
subjecting a biological sample obtained from a subject to a gene expression analysis, wherein the gene expression analysis comprises determining expression levels in the biological sample of at least two miRNAs selected from Table 6; and
using the expression levels to assist in determining the likelihood that the subject has lung cancer.

2. The method of claim 1, wherein the step of determining comprises transforming the expression levels into a lung cancer risk-score that is indicative of the likelihood that the subject has lung cancer.

3. The method of claim 2, wherein the lung cancer risk-score is the combination of weighted expression levels.

4. The method of claim 3, wherein the lung cancer risk-score is the sum of weighted expression levels.

5. The method of claim 3, wherein the expression levels are weighted by their relative contribution to predicting increased likelihood of having lung cancer

6-11. (canceled)

12. The method of claim 1 further comprising creating a report summarizing the results of the gene expression analysis.

13. The method of claim 1 further comprising creating a report that indicates the lung cancer risk-score.

14. The method of claim 1, wherein the biological sample is obtained from the respiratory epithelium of the subject.

15. The method of claim 14, wherein the respiratory epithelium is of the mouth, nose, pharynx, trachea, bronchi, bronchioles, or alveoli.

16. The method of claim 1, wherein the biological sample is obtained using bronchial brushings, broncho-alveolar lavage, or a bronchial biopsy.

17-18. (canceled)

19. The method of claim 1, wherein the at least two miRNAs are selected from the group consisting of: hsa-miR-210; hsa-miR-378; hsa-miR-221*; hsa-miR-320b; hsa-miR-1226*; hsa-miR-744; hsa-miR-320a; hsa-miR-1243; hsa-miR-345; and hsa-miR-200b.

20. The method of claim 1, wherein the at least two miRNAs are selected from the group consisting of: hsa-miR-210; hsa-miR-378; hsa-miR-221*; hsa-miR-320b; and hsa-miR-1226*.

21. The method of claim 1, wherein the at least two miRNAs are selected from the group consisting of: hsa-miR-210; hsa-miR-378; and hsa-miR-221*.

22. The method of claim 1, wherein the gene expression analysis comprises determining the expression levels in the RNA sample of at least five miRNAs selected from Table 6.

23. (canceled)

24. The method of claim 1, wherein the expression levels are determined using a quantitative reverse transcription polymerase chain reaction, a bead-based nucleic acid detection assay or a oligonucleotide array assay.

25-26. (canceled)

27. The method of claim 1, wherein the lung cancer is a adenocarcinoma, squamous cell carcinoma, small cell cancer or non-small cell cancer.

28. A computer implemented method for processing genomic information, the method comprising:
obtaining data representing expression levels in a biological sample of at least two miRNAs selected from Table 6, wherein the biological sample was obtained of a subject; and
using the expression levels to assist in determining the likelihood that the subject has lung cancer.

29. The computer implemented method of claim 28, wherein the step of determining comprises calculating a risk-score indicative of the likelihood that the subject has lung cancer.

30. The computer implemented method of claim 29, wherein computing the risk-score involves determining the combination of weighted expression levels, wherein the expression levels are weighted by their relative contribution to predicting increased likelihood of having lung cancer.

31-32. (canceled)

33. The computer implemented method of claim 28, wherein the at least two miRNAs are selected from the group consisting of: hsa-miR-210; hsa-miR-378; hsa-miR-221*; hsa-miR-320b; hsa-miR-1226*; hsa-miR-744; hsa-miR-320a; hsa-miR-1243; hsa-miR-345; and hsa-miR-200b.

34-56. (canceled)
